(19) United States
(12) Patent Application Publication
(10) Pub. No.: US 2023/0117083 A1
(43) Pub. Date: Apr. 20, 2023

(54) METHODS OF NUCLEIC ACID SYNTHESIS

(71) Applicant: Nuclera Nucleics Ltd., Cambridge (GB)

(72) Inventors: Tobias William Barr Ost, Cambridge (GB); Gordon Ross McInroy, Cambridge (GB); Zachary Beyer Gaber, Cambridge (GB); Harold Swerdlow, Cambridge (GB); Cecilia Tognoloni, Cambridge (GB)

(21) Appl. No.: 17/794,320

(22) PCT Filed: Jan. 22, 2021
(86) PCT No.: PCT/GB2021/050154

§ 371 Date: Jul. 21, 2022

(30) Foreign Application Priority Data
Jan. 22, 2020 (GB) 2000902.3
Aug. 21, 2020 (GB) 2013102.5

(51) Int. Cl. C12P 19/34; C12N 9/24

(52) U.S. Cl. CPC C12P 19/34; C12N 9/2497; C12Y 302/02027

(57) ABSTRACT

The invention relates to improved methods of enzymatic solid-supported nucleic acid synthesis that make use of terminal deoxynucleotidyl transferase (TdT) enzymes or modified terminal deoxynucleotidyl transferase (TdT) enzymes on polyacrylamide type supports. The invention further relates to the use of kits comprising said enzymes in a method of solid-supported nucleic acid synthesis.

### FIELD OF THE INVENTION

The invention relates to methods of enzymatic solid-supported nucleic acid synthesis that make use of terminal deoxynucleotidyl transferase (TdT) enzymes. The invention further relates to the use of kits comprising said enzymes in a method of solid-supported nucleic acid synthesis.

### BACKGROUND OF THE INVENTION

Nucleic acid synthesis is vital to modern biotechnology. The rapid pace of development in the biotechnology arena has been made possible by the scientific community’s ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is practically impossible to synthesise a DNA strand greater than 200 nucleotides in length with a desirable yield, and most DNA synthesis companies only offer up to 120 nucleotides. In comparison, an average protein-coding gene is of the order of 2000- 3000 contiguous nucleotides, a chromosome is at least a million contiguous nucleotides in length and a eukaryotic genome can be in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be readily synthesised beyond 200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3ʹ-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single stranded DNA synthesis, as opposed to controlled, takes advantage of TdT’s deoxynucleoside triphosphate (dNTP) 3ʹ tailing properties on single-stranded DNA to create, for example, homopolymeric adaptor sequences for next-generation sequencing library preparation. In controlled extensions, a reversible deoxynucleoside triphosphate termination technology needs to be employed to prevent uncontrolled addition of dNTPs to the 3ʹ-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents.

However, TdT has not been shown to efficiently add nucleoside triphosphates containing 3ʹ-O-reversibly terminating moieties for building up a nascent single-stranded DNA chain necessary for a de novo synthesis cycle. A 3ʹ-O- reversible terminating moiety would prevent a terminal transferase like TdT from catalysing the nucleotide transferase reaction between the 3ʹ-end of a growing DNA strand and the 5ʹ-triphosphate of an incoming nucleoside triphosphate.

There is therefore a need to identify modified terminal deoxynucleotidyl transferases that readily incorporate 3ʹ-O- reversibly terminated nucleotides. Said modified terminal deoxynucieotidyl transferases can be used to incorporate 3ʹ-O- reversibly terminated nucleotides in a fashion useful for biotechnology and single-stranded DNA synthesis processes in order to provide an improved method of nucleic acid synthesis that is able to overcome the problems associated with currently available methods.

Solid-phase techniques are widely used in peptide synthesis, oligonucleotide synthesis, oligosaccharide synthesis and combinatorial chemistry. Solid-phase synthesis is carried out on a solid support which is held in such a way as to enable all reagents and solvents to pass through freely during a reaction. Solid-phase synthesis has a number of advantages over solution synthesis, including:
large excesses of solution-phase reagents can be used to drive reactions quickly to completion.
impurities and excess reagents are washed away and no purification is required after each step.
the process is amenable to automation on computer-controlled solid-phase synthesizers.

Methods of coating surfaces are described in for example WO2005/065814A1 and WO2013184796A1. WO2005/065814A1 describes a support modified by in-situ polymerization, and WO2013184796A1 described a support which is modified by covalent attachment of a polymer.

Solid supports (or resins) are insoluble particles to which an oligonucleotide may be bound during the nucleic acid synthesis process. A number of solid support surface types and materials are known in the art. Methods of enzymatic solid-supported nucleic acid synthesis known in the art often lead to poor yield thus limiting the number of nucleotides that can be successfully synthesised. There therefore exists a need for methods of enzymatic solid-supported nucleic acid synthesis that are amenable to high-yielding nucleic acid synthesis.

### BRIEF DESCRIPTION OF THE FIGURES

### Annotated PAGE Gel Image for N+1 Addition Reactions

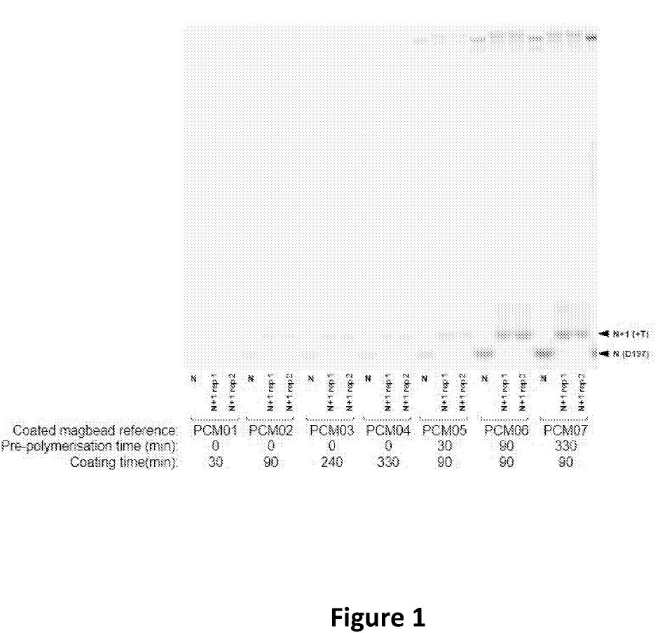

FIG. 1 shows denaturing polyacrylamide gel electrophoresis (PAGE) analysis of oligonucleotides prepared using the methods of the invention. The importance of polymer preparation is clearly shown.

### Variation in Oligo Loading per Bead Type

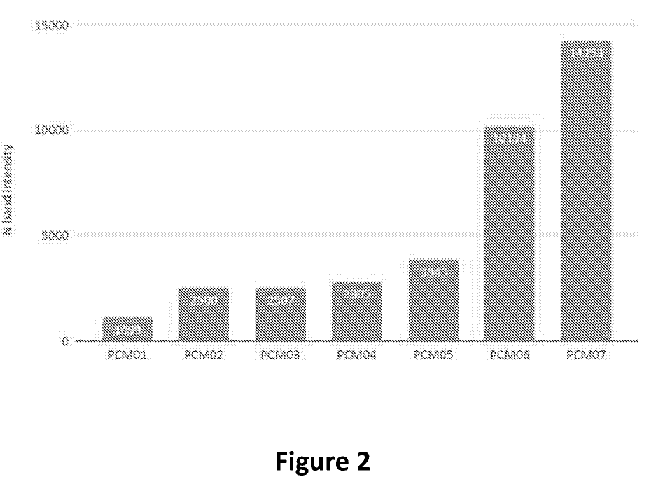

FIG. 2 shows a quantitative analysis of the PAGE image in FIG. 1.

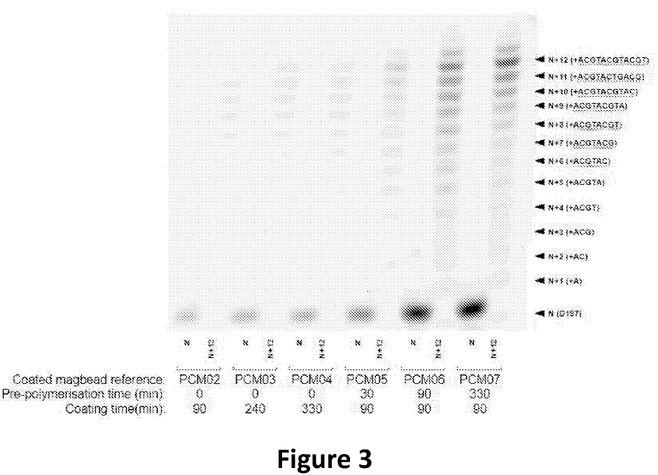

FIG. 3 shows denaturing polyacrylamide gel electrophoresis (PAGE) analysis of oligonucleotides.

FIG. 3 shows the PAGE image for N+12 addition reactions performed on different PAC-BRAC polymer coated bead types (PCM02-07).

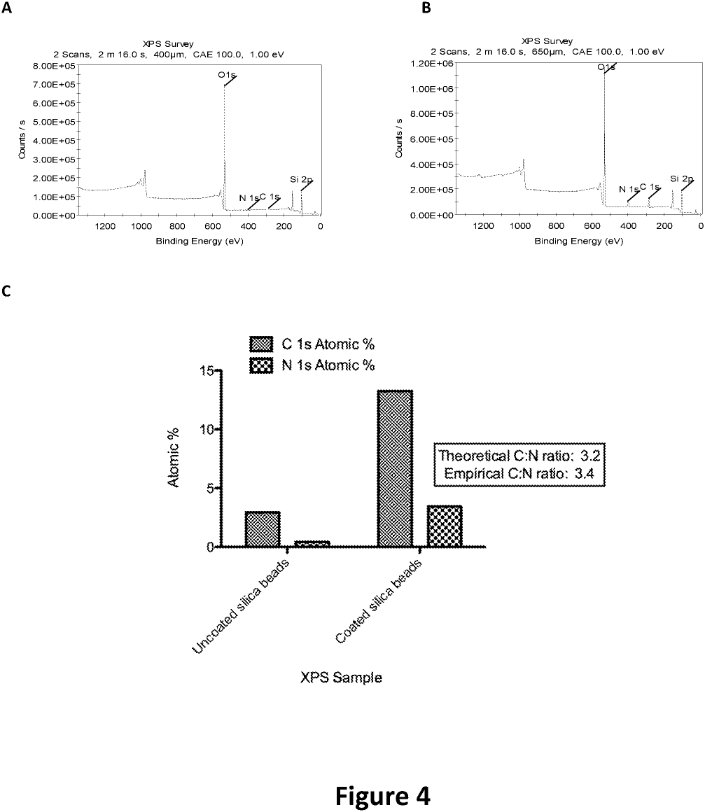

FIG. 4. X-ray photoelectron spectroscopy (XPS) of coated and uncoated silica beads. PAC-BRAC polymerisation and coating was performed in situ with silica paramagnetic particles. Subsequently, X-ray photoelectron spectroscopy (XPS) was used to analyse the surface chemistry of silica beads with (Panel B) and without (Panel A) a PAC-BRAC coating. The atomic % of oxygen (O), nitrogen (N), carbon (C), and silicon (Si) was determined and used to establish the presence of a polyacrylamide coating on the surface of the silica paramagnetic particles. Comparing the atomic % of carbon and nitrogen between the uncoated and coated samples (Panel C), as well as the ratio of carbon and nitrogen, clearly shows the presence of a polyacrylamide-based surface coating.

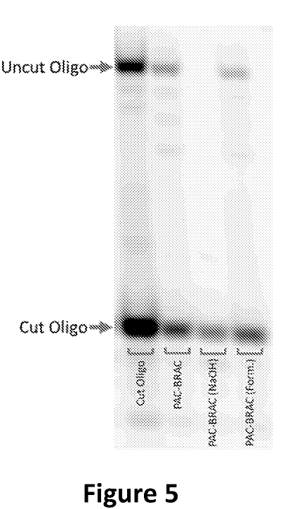

FIG. 5. Denaturing polyacrylamide gel electrophoresis analysis of oligonucleotides prepared using the methods of the invention. Cleaned silica paramagnetic particles were coated with pre-polymerised PAC-BRAC for ninety minutes with periodic vortexing. The coated particles were washed with 10 mM potassium phosphate pH 7. A phosphorothioate-containing oligonucleotide of SEQ 2 was coupled to the PAC-BRAC surface in a 60 minute incubation at 52° C. The oligonucleotide-coated particles were split into three aliquots and washed with different solutions to remove non-specifically bound oligonucleotide. Finally, the particles were exposed to a solution containing uracil DNA glycosylase (UDG) and N,N′-dimethylethylenediamine (DMED) to cleave the oligonucleotide at the U base. Subsequent analysis was via denaturing polyacrylamide gel electrophoresis, which was visualised by virtue of the cyanine 3 (cy3) dye present in the oligonucleotide.

Lane 1: Control lane. Oligonucleotide was exposed to the UDG/DMED cleavage solution. The higher molecular weight band is uncut oligonucleotide and the lower molecular weight band is cut oligonucleotide. Lane 2: PAC-BRAC beads washed with 1 M aqueous sodium chloride, 0.1 % tween-20, and 20 mM HEPES KOH. The presence of bands in the gel shows that DNA was bound to the particles. The presence of full-length (uncut) oligonucleotide suggests some non-specific binding was present. Lane 3: PAC-BRAC beads washed with 20 mM aqueous sodium hydroxide. The presence of bands in the gel shows that DNA was bound to the particles. The absence of any full-length (uncut) oligonucleotide demonstrates that no non-specific binding was present. Lane 4: PAC-BRAC beads washed with 50% formamide. The presence of bands in the gel shows that DNA was bound to the particles. The presence of full-length (uncut) oligonucleotide suggests some non-specific binding was present, though at a reduced level to that seen in Lane 1.

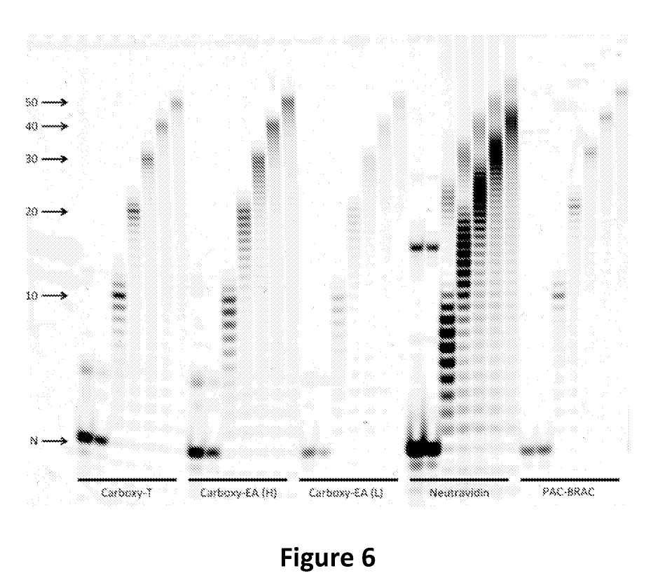

FIG. 6. Denaturing polyacrylamide gel electrophoresis (PAGE) analysis of DNA products synthesised from a selection of solid supports. DNA synthesis was performed enzymatically using an engineered terminal deoxynucleotidyl transferase (TdT) to incorporate reversibly terminated nucleotides into a growing single stranded DNA (ssDNA) product. For each surface, 0, 10, 20, 30, 40, and 50 cycles of synthesis were performed in separate experiments. Synthesis initiators were grafted to the solid supports as follows: (1) 1-Ethyl-3-(3-dimethylaminopropyl)carbodiimide (EDC) coupling to carboxyl surface, followed by capping with tris(hydroxymethyl)aminomethane (Carboxy-T) or ethanolamine (Carboxy-EA); (2) affinity interaction between a biotinylated initiator and a neutravidin surface (Neutraividin); (3) reaction between a phosphorothioate-containing initiator and a haloacetimide containing surface (PAC-BRAC). (H) and (L) indicate high and low initiator loading densities respectively. The synthesis process is markedly more efficient when performed on the PAC-BRAC surface, as evidenced by the primary product being the target product in all experiments (in contrast to other surfaces) and the significant reduction in N-1, N-2, etc deletion products.

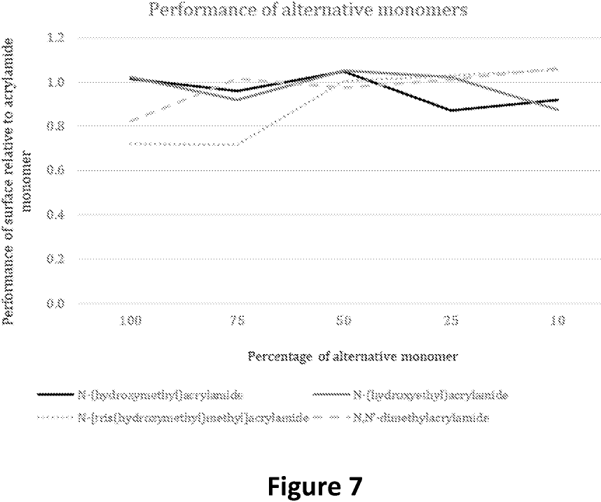

FIG. 7. The effect of first co-monomer identity on polymer performance. xPAC-BRAC (where xPAC indicates that the first co-monomer was varied) was-pre-polymerised and coated onto silica paramagnetic particles. The first co-monomer was selected from acrylamide, N-(hydroxylmethyl)acrylamide, N-(hydroxyethyl)acrylamide, N,N′-dimethyl acrylamide, and N-[tris(hydroxymethyl)methyl]acrylamide. At 100% alternative monomer, there was complete replacement of acrylamide, while at 50% alternative monomer 50% acrylamide was present. Synthesis initiators were then grafted to the polymer and 17 cycles of DNA synthesis were performed using an engineered terminal deoxynucleotidyl transferase and reversibly terminated nucleotides. The synthesised DNA was then cleaved from the solid support and prepared into a library and sequenced on an Illumina iSeq. The percentage of reads with the correct length and sequence identity was calculated for each sample. All samples were then normalised to the value obtained when using acrylamide as the first co-monomer. The data clearly shows that the identity of the first co-monomer can be varied to alter the properties of the polymer coating on a solid support. Indeed, certain compositions exceed the performance obtained when using acrylamide as the first co-monomer. Note that values shown are the average of two technical duplicates.

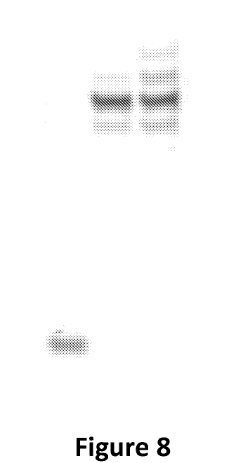

FIG. 8. Denaturing polyacrylamide gel electrophoresis (PAGE) analysis of oligonucleotides prepared using the methods of the invention in a frit-retained beads format. Polymer coated beads were prepared using the method described herein. Beads were loaded into either wells of a 96-well plate with a 0.22 micron frit or 384-well plate with a 0.45 micron frit (Millipore HTS plates). DNA synthesis was performed enzymatically using an engineered terminal deoxynucleotidyl transferase (TdT) to incorporate reversibly terminated nucleotides into a growing single stranded DNA (ssDNA) product. Introduction of addition solution (NAM), wash solution, nitrite deblock solution (NDS) was accomplished by dispensing liquid into the wells with a liquid handling robot and removal of solution was accomplished by application of a vacuum to draw liquid through the filter frit of the plate. The polymer coated silica particles were retained in the wells due to their size (> 1 micron) being greater than the frit pore size of 0.22 micron or 0.45 micron. Lane 1 of the gel shows the DNA initiator (N); lane 2 shows the product after 8 cycle of enzymatic DNA synthesis (N+8) on a 96-well plate with 0.22 micron frit; lane 3 shows the product after 8 cycles of enzymatic DNA synthesis (N+8) on a 384-well plate with 0.45 frit. The major product in lanes 2 and 3 is the N+8 oligonucleotide. The fainter bands below (N+7) and above (N+8) arise due to incomplete enzymatic addition and multiple addition within one cycle respectively.

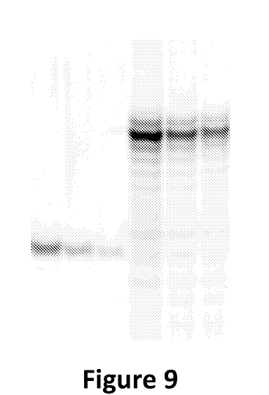

FIG. 9. Denaturing polyacrylamide gel electrophoresis (PAGE) analysis of oligonucleotides prepared using the methods of the invention in a polymer coated frit format. The glass fibre frits of a multiwell plate were cleaned with 80% Decon, 1 M NaOH, and 0.1M HCl in sequence. The frit was coated with prepolymerised PAC-BRAC polymer by repeated 60 second incubations and vacuum solution removal. Oligonucleotide with phosphorothioate linkages was then grafted to the polymer to act as an initiator for enzymatic DNA synthesis. DNA synthesis was performed enzymatically using an engineered terminal deoxynucleotidyl transferase (TdT) to incorporate reversibly terminated nucleotides into a growing single stranded DNA (ssDNA) product. Introduction of addition solution (NAM), wash solutions, and deblock solution (NDS) was accomplished by dispensing liquid into the wells with a liquid handling robot and removal of solutions was accomplished by application of a vacuum to draw liquid through the polymer coated frit of the plate. Synthesised oligonucleotide was cleaved from the polymer coated frit and analysed by polyacrylamide gel electrophoresis (PAGE). Lanes 1-3 contain DNA initiator (N); lanes 4-6 contain the products after 8 cycles of enzymatic DNA synthesis. The major product in lanes 4-6 is N+8 corresponding to the full-length synthesis product.

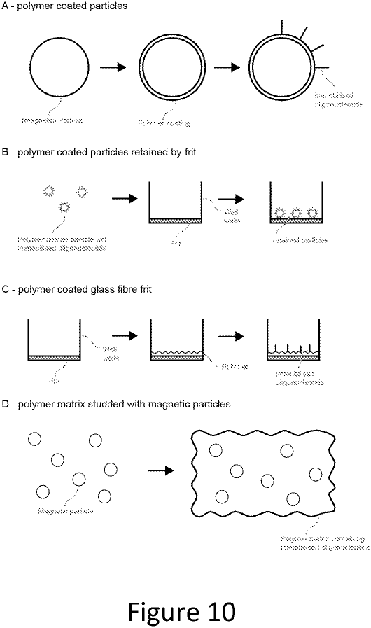

FIG. 10. Schematic showing various types of solid support

Part A shows coated particles. The particles are optionally magnetic.

Part B shows the particles retained in a frit.

Part C shows a coated frit.

Part D shows a plurality of coagulated particles.

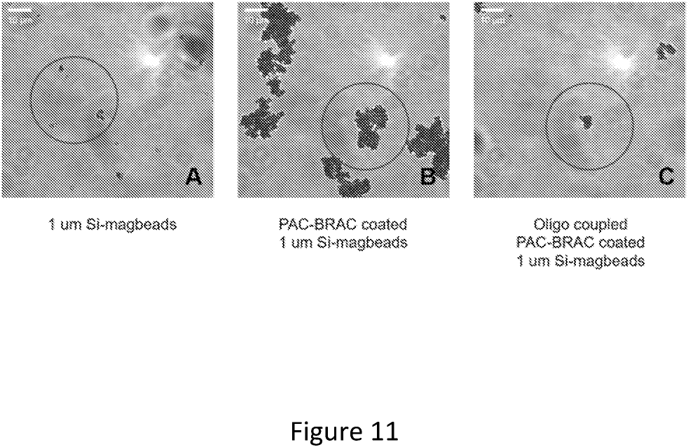

FIG. 11. Particle coagulation The same volume (10 µL) of 1 mg/mL magbead suspensions (in 20 mM HEPES 50 mM NaCl 0.1% v/v Tween 20 pH 7.2 buffer) were applied to the surface of a glass microscope slide and sealed with a glass cover slip. Slides were prepared using suspensions of uncoated (1 µm silica coated paramagnetic particles, panel A), polymer-coated (PAC-BRAC, Panel B) and oligo-coupled (oligo = 43 mer with 5ʹ phosphorothioate conjugation modification, Panel C) magbeads were taken on a microscope (x25 objective). An approximate 10 µm scale bar is given in each panel for reference. A circle has been drawn on each panel as a region of interest to highlight populations of particles. The uncoated particles are generally monodisperse and form a well separated suspension. Coated beads form large multi-particle aggregates of heterogeneous size typically >50 µm. Oligo coupled bead suspensions appear to exist in smaller aggregated populations, within a nominal size range of 10-50 µm. This data demonstrates the effect polymer coating and oligo coupling to the particle surface has on their aggregative behaviour.

### SUMMARY OF THE INVENTION

In the process of testing suitable solid supports for TdT based extension reactions, the inventors have identified particular supports which are surprisingly advantageous in terms of the efficiency of extension. The inventors have identified methods of enzymatic solid-supported nucleic acid synthesis that offer improved yield and specificity for target products relative to methods known in the art. The inventors have identified solid support coatings for use in enzymatic solid-supported nucleic acid synthesis, specifically where the enzyme is terminal deoxynucleotidyl transferase (TdT) enzyme or modified terminal deoxynucleotidyl transferase (TdT) enzyme. The methods described herein result in a significant reduction of the formation of deletion products (N-1, N-2, etc.). Thus the invention described herein relates to improved methods of enzymatic solid-supported nucleic acid synthesis where the enzyme is terminal deoxynucleotidyl transferase (TdT) enzyme or modified terminal deoxynucleotidyl transferase (TdT) enzyme. Data described herein shows that the claimed surfaces result in a significant reduction of the formation of deletion products (N-1, N-2, etc.).

Described herein is a method of nucleic acid synthesis, wherein the method comprises the steps of:
(a) providing a solid support, wherein the solid support is coated with a co-polymer to which an initiator oligonucleotide is attached, wherein the co-polymer is a co-polymer of a first co-monomer selected from acrylamide, methacrylamide, N-methylacrylamide, N,N′-dimethylacrylamide, hydroxyethyl methacrylate and N-vinyl pyrrolidinone and a second co-monomer which attaches the initiator; and
(b) adding a 3’-blocked nucleoside triphosphate to said initiator oligonucleotide in the presence of a terminal deoxynucleotidyl transferase (TdT) or modified terminal deoxynucleotidyl transferase (TdT) enzyme.

Disclosed herein are supports coated with a pre-polymerised mixture of monomers. The pre-polymerised mixtures are linear polymers having no bifunctional cross linking moieties. The supports are coated with the polymerized mixtures, no chemical pre-treatment or covalent attachment is required. The supports may be a plurality of discreet particles or a planar surface. The support may be a coagulated plurality of particles.

Disclosed is a method of nucleic acid synthesis, wherein the method comprises the steps of:
(a) providing a solid support comprising a plurality of particles coated with a pre-polymerised material, wherein the pre-polymerised material comprises a co-polymer to which an initiator oligonucleotide is attached or is to be attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches to or is attached to the initiator;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent; and
(d) repeating steps (b) and (c) to synthesise an extended nucleic acid.

Disclosed is a method of nucleic acid synthesis, wherein the method comprises the steps of:
(a1) providing a solid support comprising particles coated with a pre-polymerised material, wherein the pre-polymerised material comprises a co-polymer to which an initiator oligonucleotide is to be attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches to the initiator;
(a2) coupling an initiator oligonucleotide to said co-polymer to form a solid-supported initiator oligonucleotide;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent; and
(d) repeating steps (b) and (c) to synthesise an extended nucleic acid.

The support can comprise a plurality of particles held within the pre-polymerised material. The particles may be magnetic.

Described herein is a method of nucleic acid synthesis, wherein the method comprises the steps of:
(a) providing a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches to or is attached to the initiator;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent; and
(d) repeating steps (b) and (c) to synthesise an extended nucleic acid.

Described herein is a method of nucleic acid synthesis, wherein the method comprises the steps of:
(a) providing a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches to or is attached to the initiator;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent;
(d) repeating steps (b) and (c) to synthesise an extended nucleic acid; and
(e) detaching the extended nucleic acid from the solid support.

Described herein is a method of nucleic acid synthesis, wherein the method comprises the steps of:
(a1) providing a solid support, wherein the solid support comprises a coating of a co-polymer of one or more first co-monomer(s) and a second co-monomer, wherein the first co-monomer is acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate or N-vinyl pyrrolidinone and the second co-monomer enables coupling of an initiator oligonucleotide;
(a2) coupling an initiator oligonucleotide to said co-polymer to form a solid-supported initiator oligonucleotide.

The solid support coatings described herein comprise a co-polymer to which an initiator oligonucleotide is attached. Said coatings, when applied to solid supports may create a polymer surface which is effectively immobilised to the solid support. In the methods of the invention nucleic acid synthesis is carried out at the attached initiator oligonucleotide by addition of a 3ʹ-blocked nucleoside triphosphate in the presence of a terminal deoxynucleotidyl transferase (TdT) enzyme. The methods described herein are not limited by the format, size, geometry or the material of the solid support itself. The polymeric coatings described herein may be applied to the solid support by any method, including those known in the art. Examples of coatings that may be suited to the methods described herein are described in WO2005/065814.

Following the extension, the blocking group may be cleaved. Thus included is a method further comprising: (c) cleaving the blocking group from the 3ʹ-blocked nucleoside triphosphate in the presence of a cleaving agent.

Cleavage releases a 3ʹhydroxyl suitable for further addition/extension. The adding step and cleaving steps may be repeated in order to elongate the synthesis product. Thus included is a method wherein further nucleotides are added by repeating steps (b) and (c).

The initiator oligonucleotide is attached via polymerization or coupled after polymerisation of the two or more co-monomers. Thus included is a method wherein step (a) comprises the steps of:
(a1) providing a solid support, wherein the solid support is coated with a co-polymer of a first co-monomer and a second co-monomer, wherein the first co-monomer is acrylamide, methacrylamide, N-methylacrylamide, N,N′-dimethylacrylamide, hydroxyethyl methacrylate or N-vinyl pyrrolidinone and the second co-monomer to enables coupling of an initiator oligonucleotide;
(a2) coupling an initiator oligonucleotide to said co-polymer to form a solid-supported initiator oligonucleotide.

The first co-monomer has a single reactive point, usually a double bond for polymerisation. The first co-monomer may be one or more of acrylamide, methacrylamide, N-methylacrylamide, N,N′-dimethylacrylamide, hydroxyethyl methacrylate and/or N-vinyl pyrrolidinone. The first co-monomer can be acrylamide. The first co-monomer can be mixture of monomers such as for example acrylamide and methacrylamide.

The second co-monomer typically contains multiple reactive sites, usually both a double bond and a reactive site suitable for attachment to an initiator oligonucleotide. The reactive moiety can be selected from haloacetamide, carboxylic acid, alkyne, azide, amine or thiol. The second co-monomer can be a haloacetamide-containing monomer. The second co-monomer can be a bromoacetamide-containing monomer. Alternatively in some embodiments the second co-monomer can contain the initiator oligonucleotide having a suitable double bond.

The second co-monomer can be a monomer of formula (4a):

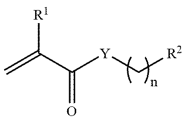

wherein R¹ is H or CH₃;

R² is an oligonucleotide or NHCOCH₂X;

Y is NR³or 0;

n is 1 to 50;

X is Cl, Br, or I;

R³ is H or an optionally substituted C₁₋₅ alkyl group.

Particularly R¹ can be H.

Particularly R² can be NHCOCH₂Br.

Particularly Y can be NH.

Particularly n can be 5.

The second co-monomer can be N-(5-bromoacetamidylpentyl) acrylamide (BRAC).

In some embodiments a portion of the second co-monomer can be attached to the initiator. Thus the second co-monomer can be an oligonucleotide comprising one or more C═C double bonds. The second co-monomer can comprise an oligonucleotide bearing a 5ʹ methacrylate moiety.

The methods described herein are not limited by the format, size, geometry or the material of the solid support itself. The solid support can comprise silica beads, paramagnetic beads, superparamagnetic beads, glass fibres, a glass slide, a plastic well or a plastic slide. The beads can be paramagnetic beads. The beads can be silica beads. The beads can comprise a silica shell. The beads can comprise superparamagnetic cores covered by silica shells. The solid support can be synthesised using the polymerised monomer mixture described herein.

Where the coating is applied to an existing surface, the surface coating can be applied as a pre-polymerised polymer mixture of the first and second co-monomers. Alternatively the surface coating can be applied to the solid support during polymerisation of the first and second co-monomers. The pre-polymerisation can be carried out for at least 90 minutes prior to exposure to the surface being coated.

The initiator oligonucleotide is attached to the support via a suitable modification to react with the reactive moiety present on the co-polymer. The modification on the initiator oligonucleotide depends on the nature of the reactive moiety present on the co-polymer. In order to react with a haloacetamide, the initiator oligonucleotide contains and is coupled via coupled via a phosphorothioate moiety, thus becoming attached via a P-S-CH₂ linkage.

Alternatively click chemistry can be used. In which case the initiator oligonucleotide is coupled via click chemistry between an azide and an alkyne.

Alternatively an amine can be reacted with a carboxyl moiety to form an amide linkage. In which case the initiator oligonucleotide is coupled via amide formation between an amine and a carboxyl moiety. This reaction is typically conducted in the presence of an activating agent such as 1-ethyl-3-(3-dimethylaminopropyl)carbodiimide (EDC).

Alternatively the oligonucleotide can contain a moiety such as methylacrylate that can be used to attach the oligonucleotide as part of the polymerization process. Thus the initiator can be attached as part of the polymerization process rather than as an additional attachment step after the polymerisation.

After synthesis of the nucleic acid is completed, the material may be detached from the solid support. The initiator sequence can contain a moiety that allows cleavage, such as a non-canonical nucleotide base. The initiator can contain a nucleotide having a base such as uracil or 8-oxo-guanine thereby allowing strand cleavage via exposure to an appropriate glycosylase.

Cycles of extension can use a 3ʹ-blocked nucleotide in order to prevent multiple extensions per cycle. The 3ʹ-blocked nucleoside triphosphate can be blocked with a group selected from 3ʹ-O-azidomethyl, 3ʹ-aminooxy, 3ʹ-O-allyl, 3ʹ-O-cyanoethyl, 3ʹ-O-acetyl, 3ʹ-O-nitrate, 3ʹ-O-phosphate, 3ʹ-O-acetyl levulinic ester, 3ʹ-O-tert butyl dimethyl silane, 3ʹ-O-trimethyl(silyl)ethoxymethyl, 3ʹ-O-ortho-nitrobenzyl, or 3ʹ-O-para-nitrobenzyl.

Suitable enzymes are described below. Any enzyme having template independent polymerase extension activity can be used. A suitable template independent polymerase is terminal deoxynucleotidyl transferase (TdT). The terminal deoxynucleotidyl transferase (TdT) enzyme can be a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species. The modification can be selected from one or more of the amino acid regions WLLNRLINRLQNQGILLYYDI, VAIF, EDN, MGA, ENHNQ, FMRA, HAl, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

The cleavage agent depends on the nature of the blocking moiety. The cleaving agent can be tris(2-carboxyethyl)phosphine (TCEP), a palladium complex, an organic or inorganic base, sodium nitrite or a photoactivated transition metal complex.

In order to unblock a 3ʹ-azidomethyl moiety the cleaving agent might be tris(2-carboxyethyl) phosphine (TCEP). In order to unblock a 3ʹ-aminoxy moiety the cleaving agent might be sodium nitrite. In order to unblock a 3ʹ-allyl moiety the cleaving agent might be a palladium complex.

Also included is a kit comprising:
(i) a solid support, wherein the solid support is coated with a co-polymer to which an initiator oligonucleotide is attached, wherein the co-polymer is a co-polymer of a first co-monomer selected from acrylamide, methacrylamide, N-methylacrylamide, N,N′-dimethylacrylamide, hydroxyethyl methacrylate and N-vinyl pyrrolidinone and a second co-monomer which attaches the initiator oligonucleotide;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

The support can comprise a plurality of magnetic particles held within a polymerised material.

Also included is a kit comprising:
(i) a solid support comprising a plurality of particles coated with a pre-polymerised material, wherein the pre-polymerised material comprises a co-polymer to which an initiator oligonucleotide is attached or is to be attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches to or is attached to the initiator; and
(ii) a 3ʹ-blocked nucleoside triphosphate.

Also included is a kit comprising:
(i) a solid support, wherein the solid support comprises a plurality of magnetic partcles held within a co-polymer to which an initiator oligonucleotide is attached, wherein the co-polymer is a co-polymer of a first co-monomer selected from acrylamide, methacrylamide, N-methylacrylamide, N,N′-dimethylacrylamide, hydroxyethyl methacrylate and N-vinyl pyrrolidinone and a second co-monomer which attaches the initiator oligonucleotide;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

Also disclosed is a kit comprising:
(i) a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches the initiator;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

Also disclosed is a kit comprising:
(i) a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches to or is attached to the initiator;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

### DETAILED DESCRIPTION OF THE INVENTION

Described herein is a method of nucleic acid synthesis, wherein the method comprises the steps of:
(a) providing a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches to or is attached to the initiator; and
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme.

Inventors have appreciated that the process of coating surfaces can be improved by
a) using a non cross-linked ‘linear’ polymer
b) not covalently attaching the polymer to the support
c) pre-polymerising the material before exposing the polymerized material to the support.

Inventors have further appreciated that whilst coating a population of beads with polymer, discreet units of solid support can be obtained, each containing a plurality of the smaller beads. The support can comprise a plurality of optionally magnetic particles held within a polymerised material. Magnetic beads of for example 1 µm in size can be bound together within a polymerized material, thereby forming discreet magnetic particles. The size of, and number of magnetic particles within, the aggregated solid supports can be controlled by the polymerization conditions, including choice of monomers, concentration of monomers and length or polymerization time. For example the solid support may contain at least 10 magnetic particles or at least 100 magnetic particles.

The solid support may have an average particle diameter of at least 50 µm. The magnetic particles within the solid support have be beads of size approximately 1 µm diameter. The magnetic particles within the solid support have be beads having an average size distribution of less than 2 µm.

Described herein is a method of nucleic acid synthesis, wherein the method comprises the steps of:
(a) providing a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate and N-vinyl pyrrolidinone and a second co-monomer which attaches the initiator; and
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme.

The method may further comprise the step:

(c) cleaving the blocking group from the 3ʹ-blocked nucleoside triphosphate in the presence of a cleaving agent.

In the methods described herein, further nucleotides may be added by repeating steps (b) and (c).

Thus the methods of the invention may comprise the steps of:
(a) providing a solid support, wherein the solid support is coated with a co-polymer to which an initiator oligonucleotide is attached, wherein the co-polymer is a co-polymer of a first co-monomer selected from acrylamide, methacrylamide, N-methylacrylamide, N,N′-dimethylacrylamide, hydroxyethyl methacrylate and N-vinyl pyrrolidinone and a second co-monomer which attaches the initiator;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent;
(d) repeating steps (b) and (c) one or more times.

In the methods described herein, step (a) may comprise the steps of:
(a1) providing a solid support, wherein the solid support is coated with a co-polymer of a first co-monomer and a second co-monomer, wherein the first co-monomer is acrylamide, methacrylamide, N-methylacrylamide, N,N′-dimethylacrylamide, hydroxyethyl methacrylate or N-vinyl pyrrolidinone and the second co-monomer enables coupling of an initiator oligonucleotide;
(a2) coupling an initiator oligonucleotide to said co-polymer to form a solid-supported initiator oligonucleotide.

Thus the methods of the invention may comprise the steps of:
(a1) providing a solid support, wherein the solid support is coated with a co-polymer of a first co-monomer and a second co-monomer, wherein the first co-monomer is acrylamide, methacrylamide, N-methylacrylamide, N,N′-dimethylacrylamide, hydroxyethyl methacrylate or N-vinyl pyrrolidinone and the second co-monomer enables coupling of an initiator oligonucleotide;
(a2) coupling an initiator oligonucleotide to said co-polymer to form a solid-supported initiator oligonucleotide;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent;
(d) repeating steps (b) and (c) one or more times.

In some of the methods described herein, the solid support coatings comprise a co-polymer to which an initiator oligonucleotide is attached. Said coatings, when applied to solid supports may create a polymer surface which is effectively immobilised to the solid support. The coatings comprise a co-polymer of one or more first co-monomers and a second co-monomer, wherein the first co-monomer may be selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, bisacrylamide, hydroxyethyl methacrylate or N-vinyl pyrrolidinone. The first co-monomer may be acrylamide. In some embodiments linear polymers are preferred.

The second co-monomer serves to provide functionalisation in the form of a chemically reactive group on the support such that the resultant co-polymer allows coupling of the co-polymer to an initiator oligonucleotide, thereby immobilizing the initiator oligonucleotide. Thus the second co-monomer can be selected from any co-monomer that is suitable for allowing coupling of the resultant co-polymer to an initiator oligonucleotide. The second co-monomer can for example be selected from a thiol-containing monomer, an amine-containing monomer, an acid-containing monomer, a haloacetamide-containing monomer, an alkyne-containing monomer and an azide-containing monomer. The second co-monomer can be a haloacetamide-containing monomer. The second co-monomer can be a bromoacetamide-containing monomer. The second co-monomer can be N-(5-bromoacetamidylpentyl) acrylamide (BRAC). The second co-monomer may contain a reactive moiety selected from haloacetamide, carboxylic acid, alkyne, azide, amine or thiol.

The second co-monomer can be a monomer of formula (1a) or (1b):

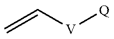

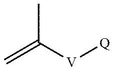

wherein Q is any group suitable to allow coupling of the resultant co-polymer to an initiator oligonucleotide;

and V is a linker group.

Alternatively Q can be attached to the initiator oligonucleotide, for example in the form of a methacrylate group.

When the second co-monomer is a monomer of formula (1a) or formula (1b), Q can be a group suitable to allow coupling of the resultant co-polymer to an initiator oligonucleotide. Q can be a group comprising an azide, haloacetamide, alkyne, amine, carboxylic acid or thiol moiety. Q can be a group comprising a haloacetamide moiety. Q can be selected from C(O)CH₂Br, NH₂, N₃, CO₂H, NHC(O)CH₂Br, C≡CH and SH. Q can be NHC(O)CH₂Br. V can be any suitable linker group. V can be selected from an optionally substituted alkyl linker, an optionally substituted alkoxy linker or an optionally substituted polyethylene glycol linker. V can be optionally substituted C₁₋₅₀ alkyl. V can be optionally substituted C(O)C₁₋₅₀ alkyl. V can be -(OCH₂CH₂)-ₙ where n is 1 to 20.

The second co-monomer can be a monomer of formula (2a) or (2b):

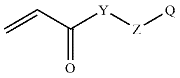

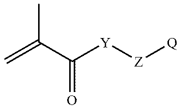

wherein Q is any group suitable to allow coupling of the resultant co-polymer to an initiator oligonucleotide;

Y is NR¹ or O;

Z is an optionally substituted C₁₋₅₀ alkyl bridge;

and R¹ is H or an optionally substituted C₁₋₅ alkyl group.

Alternatively Q can be attached to the initiator oligonucleotide, for example in the form of a methacrylate group.

When the second co-monomer is a monomer of formula (2a) or formula (2b), Q can be a group suitable to allow coupling of the resultant co-polymer to an initiator oligonucleotide. Q can be a group comprising an azide, haloacetamide, alkyne, amine, carboxylic acid or thiol moiety. Q can be a group comprising a haloacetamide moiety. Q can be selected from C(O)CH₂Br, NH₂, N₃, CO₂H, NHC(O)CH₂Br, C≡CH and SH. Q can be NHC(O)CH₂Br. V can be any suitable linker group. V can be selected from an optionally substituted alkyl linker, an optionally substituted alkoxy linker or an optionally substituted polyethylene glycol linker. V can be optionally substituted C₁₋₅₀ alkyl. V can be optionally substituted C(O)C₁₋₅₀ alkyl. V can be -(OCH₂CH₂)-ₙ where n is 1 to 20.

Y can be NH. Y can be O. R¹ can be H. R¹ can be an optionally substituted C₁₋₅ alkyl group. Z can be an optionally substituted C₁₋₂₀ alkyl bridge. Z can be an optionally substituted C₁₋₁₀ alkyl bridge. Z can be an optionally substituted C₅ alkyl bridge. Z can be a C₅ alkyl bridge.

The second co-monomer can be a monomer of formula (3) or (3a):

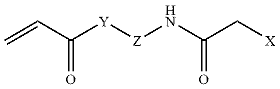

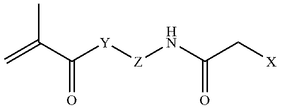

wherein X is Cl, Br or I;

Y is NR¹ or O;

Z is an optionally substituted C₁₋₅₀ alkyl bridge;

and R¹ is H or an optionally substituted C₁₋₅ alkyl group.

When the second co-monomer is a monomer of formula (3) or (3a), X can be Br. X can be Cl. X can be I. Y can be NH. Y can be O. R¹ can be H. R¹ can be an optionally substituted C₁₋₅ alkyl group. Z can be an optionally substituted C₁₋₂₀ alkyl bridge. Z can be an optionally substituted C₁₋₁₀ alkyl bridge. Z can be an optionally substituted C₅ alkyl bridge. Z can be a C₅ alkyl bridge.

The second co-monomer can be a monomer of formula (4) or (4a):

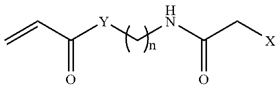

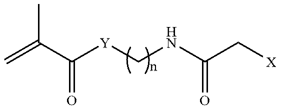

wherein X is Cl, Br or I;

Y is NR¹ or O;

Z is an optionally substituted C₁₋₅₀ alkyl bridge;

and R¹ is H or an optionally substituted C₁₋₅ alkyl group.

When the second co-monomer is a monomer of formula (4) or (4a), X can be Br. X can be Cl. X can be I. Y can be NH. Y can be O. R¹ can be H. R¹ can be an optionally substituted C₁₋₅ alkyl group. n can be 2 to 10. n can be 5.

In the methods described herein, the polymerisation reaction between the first and second co-monomer may be carried out in the presence or absence of the solid support. When carried out in the presence of the solid support, the coating is applied to the solid support as the polymerisation reaction progresses. Accordingly the coating may be applied to the solid support during polymerisation of the first and second co-monomers. Alternatively the polymerisation reaction may be carried out independently of the solid support. Therefore the resultant co-polymer may be coated on to the solid support separately to the polymerisation process. Accordingly the coating may be applied to the solid support as a pre-polymerised polymer mixture of the first and second co-monomers.

In the polymerisation reaction between the first and second co-monomer, the first co-monomer is used in a molar excess relative to the second co-monomer. For example, the second co-monomer may be present in an amount of 1 mol% or less relative to the total molar quantity of co-monomers. The second co-monomer may be present in an amount of 1 mol% or greater relative to the total molar quantity of co-monomers. The second co-monomer may be present in an amount of 2 mol% or greater relative to the total molar quantity of co-monomers.

In the methods herein, the initiator oligonucleotide is coupled to the solid support co-polymer coating to form a solid-supported initiator oligonucleotide. The initiator oligonucleotide may be coupled to the solid support co-polymer coating using any suitable method. The initiator oligonucleotide may for example be coupled to the solid support co-polymer coating via a phosphorothioate moiety or via click chemistry between an azide and an alkyne. Preferably the initiator oligonucleotide is coupled via a phosphorothioate moiety. In one embodiment the second co-monomer is a haloacetamide-containing monomer and the initiator oligonucleotide is coupled to the solid support co-polymer coating via a phosphorothioate moiety, wherein the phosphorothioate moiety couples to the co-polymer by displacement of halide from the haloacetamide-containing portion of the co-polymer.

Prior to coupling to the solid support co-polymer coating the initiator oligonucleotide may comprise a moiety of formula (5):

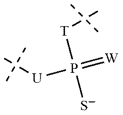

wherein, U is O, S or NR²;

T is O, S or an optionally substituted C₁₋₁₀ alkyl group;

W is O or S;

R² is H or an optionally substituted C₁₋₁₀ alkyl group.

Where the initiator oligonucleotide is coupled to the solid support co-polymer coating via a phosphorothioate moiety, prior to coupling to the solid support co-polymer coating the initiator oligonucleotide may comprise a moiety of formula:

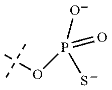

The phosphorothioate can be on the terminus of the oligonucleotide, as shown above, or can be internal to the sequence. Coupling can be performed using a moiety of formula:

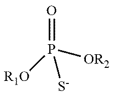

where R₁ is an oligo fragment and R² is an oligo fragment.

The methods described herein are not limited by the format, size, geometry or the material of the solid support itself. The solid support may comprise any material suited to the methods of the invention. The solid support may be a silica based solid support. The solid support may be a silica based solid support wherein said silica is fused silica. The solid support may be a polystyrene based support. The solid support may be a plastic well or a plastic slide. The solid support may comprise silica beads, magnetic beads, paramagnetic beads, superparamagnetic beads, glass fibres or a glass slide. The solid support may be silica beads. The solid support may be silica superparamagnetic beads. The solid support may be polystyrene beads. The solid support may be a non-silica based support. The solid support may comprise silica beads, paramagnetic beads, glass fibres, a glass slide, a plastic well or a plastic slide. The beads may be silica beads or comprise a silica shell.

As used herein, the term “beads” covers any particle of suitable size. The solid support particles may have a diameter within the range of 0.1-200 µm. The solid support particles may have a diameter within the range of 0.1-50 µm. The solid support particles may have a diameter within the range of 100-5000 nm.

The nucleic acid strands may include a cleavage site to enable cleavage from the solid support. The cleavage site may be a base or base sequence recognisable by an enzyme. A base recognised by an enzyme, such as a glycosylase, may be removed to generate an abasic site which may be cleaved by chemical or enzymatic means. An example of such a glycosylase system includes the presence of a uracil base in the initiator sequence, which may be excised with uracil DNA glycosylase (UDG) to leave an abasic site which may be cleaved with, for example, basic solutions, organic amines, or an endonuclease (such as endonuclease VIII), to release a nucleic acid bearing a 5ʹ-phosphate into solution. A base sequence may be recognised and cleaved by a restriction enzyme.

In the methods of the invention 3ʹ-blocked nucleoside triphosphates are added in the presence of a terminal deoxynucleotidyl transferase (TdT) enzyme which results in extension of the solid-supported sequence by one nucleotide unit. The 3ʹ-blocking group present on the 3ʹ-blocked nucleoside triphosphates prevents further incorporation of nucleotides. Upon cleavage of the blocking group present on the 3ʹ-blocked nucleoside triphosphate, the solid-supported nucleotide sequence may be further extended by adding a further 3ʹ-blocked nucleoside triphosphate in the presence of a terminal deoxynucleotidyl transferase (TdT) enzyme. Cleavage of the blocking group and addition of a further 3ʹ-blocked nucleoside triphosphate in the presence of a terminal deoxynucleotidyl transferase (TdT) enzyme may be repeated any number of times as desired to synthesise a target sequence.

The 3ʹ-blocked nucleoside 5ʹ-triphosphate can be blocked by any chemical group that can be unmasked to reveal a 3ʹ-OH. For example, the 3ʹ-blocked nucleoside triphosphate can be blocked by a 3ʹ-O-azidomethyl, 3ʹ-aminooxy, 3ʹ-O-allyl group, 3ʹ-O-cyanoethyl, 3ʹ-O-acetyl, 3ʹ-O-nitrate, 3ʹ-O-phosphate, 3ʹ-O-acetyl levulinic ester, 3ʹ-O-tert butyl dimethyl silane, 3ʹ-O-aminoxy oxime, 3ʹ-O-trimethyl(silyl)ethoxymethyl, 3ʹ-O-ortho-nitrobenzyl, and 3ʹ-O-para-nitrobenzyl. The 3ʹ-blocking group may be selected from 3ʹ-O-azidomethyl, 3ʹ-aminooxy, 3ʹ-O-cyanoethyl and a 3ʹ-O-allyl group. The 3ʹ-blocked nucleoside 5ʹ-triphosphate can also be blocked by any chemical group that can be directly utilized in chemical ligations, such as copper-catalyzed or copper-free azide-alkyne click reactions and tetrazine-alkene click reactions. The 3ʹ-blocked nucleoside triphosphate can include chemical moieties containing an azide, alkyne, alkene, and tetrazine. In another embodiment the 3ʹ-blocked nucleoside 5ʹ-triphosphate can be blocked by a chemical group that can be unmasked to reveal a 3ʹ-O-NH₂, which can subsequently be unmasked to reveal a 3ʹ-OH. The 3ʹ-blocked nucleoside triphosphate can be blocked by a 3ʹ-O-NC(CH₃)₂.

In the methods of the invention, the blocking group of the 3ʹ-blocked nucleoside triphosphate is cleaved in the presence of a cleaving agent. The cleaving agent used will depend on the 3ʹ-blocking group present and may be any cleaving agent suitable for cleaving the 3ʹ-blocking group. For instance, tris(2-carboxyethyl)phosphine (TCEP) or tris(hydroxypropyl)phosphine (THPP) can be used to cleave a 3ʹ-O-azidomethyl group, palladium complexes can be used to cleave a 3ʹ-O-allyl group, or sodium nitrite can be used to cleave a 3ʹ-aminoxy group. In one embodiment, the cleaving agent is selected from: tris(2- carboxyethyl)phosphine (TCEP), a palladium complex or sodium nitrite. The cleaving agent may be selected from tris(2-carboxyethyl)phosphine (TCEP), tris(hydroxypropyl)phosphine (THPP), a palladium complex, an organic or inorganic base, sodium nitrite and a photoactivated transition metal complex. In one embodiment the photoactivated transition metal complex is tris(2,2ʹ-bipyridyl)ruthenium(II)).

In one embodiment, the cleaving agent is added in the presence of a cleavage solution comprising a denaturant, such as urea, guanidinium chloride, formamide or betaine. The addition of a denaturant has the advantage of being able to disrupt any undesirable secondary structures in the DNA. In a further embodiment, the cleavage solution comprises one or more buffers. It will be understood by the person skilled in the art that the choice of buffer is dependent on the exact cleavage chemistry and cleaving agent required.

The methods described herein rely on terminal deoxynucleotidyl transferase (TdT) enzymes or modified terminal deoxynucleotidyl transferase (TdT) enzymes. Sequences described herein are modified from the sequence of the Spotted Gar, but the corresponding changes can be introduced into the homologous sequences from other species. Terminal transferases are ubiquitous in nature and are present in many species. Many known TdT sequences have been reported in the NCBI database htt://www.ncbi.nlm.nih.gov/. The inventors have identified a number of modified TdT enzymes with improved properties. Any such TdT enzyme or modified TdT enzyme or a truncated version thereof may be used in the methods described herein.

| | |
|---|---|
| GI Number | Species http://www.ncbi.nlm.nih.gov/ |
| gi \| 768 | Bos taurus |
| gi \| 460163 | Gallus gallus |
| gi \| 494987 | Xenopus laevis |
| gi \| 1354475 | Oncorhynchus mykiss |
| gi \| 2149634 | Monodelphis domestica |
| gi \| 12802441 | Mus musculus |
| gi \| 28852989 | Ambystoma mexicanum |
| gi \| 38603668 | Takifugu rubripes |
| gi \| 40037389 | Raja eglanteria |
| gi \| 40218593 | Ginglymostoma cirratum |
| gi \| 46369889 | Danio rerio |
| gi \| 73998101 | Canis lupus familiaris |
| gi \| 139001476 | Lemur catta |
| gi \| 139001490 | Microcebus murinus |
| gi \| 139001511 | Otolemur garnettii |
| gi \| 148708614 | Mus musculus |
| gi \| 149040157 | Rattus norvegicus |
| gi \| 149704611 | Equus caballus |
| gi \| 164451472 | Bos taurus |
| gi \| 169642654 | Xenopus (Silurana) tropicalis |
| gi \| 291394899 | Oryctolagus cuniculus |
| gi \| 291404551 | Oryctolagus cuniculus |
| gi \| 301763246 | Ailuropoda melanoleuca |
| gi \| 311271684 | Sus scrofa |
| gi \| 327280070 | Anolis carolinensis |
| gi \| 334313404 | Monodelphis domestica |
| gi \| 344274915 | Loxodonta africana |
| gi \| 345330196 | Ornithorhynchus anatinus |
| gi \| 348588114 | Cavia porcellus |
| gi \| 351697151 | Heterocephalus glaber |
| gi \| 355562663 | Macaca mulatta |
| gi \| 395501816 | Sarcophilus harrisii |
| gi \| 395508711 | Sarcophilus harrisii |
| gi \| 395850042 | Otolemur garnettii |
| gi \| 397467153 | Pan paniscus |
| gi \| 403278452 | Saimiri boliviensis boliviensis |
| gi \| 410903980 | Takifugu rubripes |
| gi \| 410975770 | Felis catus |
| gi \| 432092624 | Myotis davidii |
| gi \| 432113117 | Myotis davidii |
| gi \| 444708211 | Tupaia chinensis |
| gi \| 460417122 | Pleurodeles waltl |
| gi \| 466001476 | Orcinus orca |
| gi \| 471358897 | Trichechus manatus latirostris |
| gi \| 478507321 | Ceratotherium simum simum |
| gi \| 478528402 | Ceratotherium simum simum |
| gi \| 488530524 | Dasypus novemcinctus |
| gi \| 499037612 | Maylandia zebra |
| gi \| 504135178 | Ochotona princeps |
| gi \| 505844004 | Sorex araneus |
| gi \| 505845913 | Sorex araneus |
| gi \| 507537868 | Jaculus jaculus |
| gi \| 507572662 | Jaculus jaculus |
| gi \| 507622751 | Octodon degus |
| gi \| 507640406 | Echinops telfairi |
| gi \| 507669049 | Echinops telfairi |
| gi \| 507930719 | Condylura cristata |
| gi \| 507940587 | Condylura cristata |
| gi \| 511850623 | Mustela putorius furo |
| gi \| 512856623 | Xenopus (Silurana) tropicalis |
| gi \| 512952456 | Heterocephalus glaber |
| gi \| 524918754 | Mesocricetus auratus |
| gi \| 527251632 | Melopsittacus undulatus |
| gi \| 528493137 | Danio rerio |
| gi \| 528493139 | Danio rerio |
| gi \| 529438486 | Falco peregrinus |
| gi \| 530565557 | Chrysemys picta bellii |
| gi \| 532017142 | Microtus ochrogaster |
| gi \| 532099471 | lctidomys tridecemlineatus |
| gi \| 533166077 | Chinchilla lanigera |
| gi \| 533189443 | Chinchilla lanigera |
| gi \| 537205041 | Cricetulus griseus |
| gi \| 537263119 | Cricetulus griseus |
| gi \| 543247043 | Geospiza fortis |
| gi \| 543351492 | Pseudopodoces humilis |
| gi \| 543731985 | Columba livia |
| gi \| 544420267 | Macaca fascicularis |
| gi \| 545193630 | Equus caballus |
| gi \| 548384565 | Pundamilia nyererei |
| gi \| 551487466 | Xiphophorus maculatus |
| gi \| 551523268 | Xiphophorus maculatus |
| gi \| 554582962 | Myotis brandtii |
| gi \| 554588252 | Myotis brandtii |
| gi \| 556778822 | Pantholops hodgsonii |
| gi \| 556990133 | Latimeria chalumnae |
| gi \| 557297894 | Alligator sinensis |
| gi \| 558116760 | Pelodiscus sinensis |
| gi \| 558207237 | Myotis lucifugus |
| gi \| 560895997 | Camelus ferus |
| gi \| 560897502 | Camelus ferus |
| gi \| 562857949 | Tupaia chinensis |
| gi \| 562876575 | Tupaia chinensis |
| gi \| 564229057 | Alligator mississippiensis |
| gi \| 564236372 | Alligator mississippiensis |
| gi \| 564384286 | Rattus norvegicus |
| gi \| 573884994 | Lepisosteus oculatus |

The inventors have modified the terminal transferase from Lepisosteus oculatus TdT (spotted gar) (shown below). However the corresponding modifications can be introduced into the analogous terminal transferase sequences from any other species, including the sequences listed above in the various NCBI entries. Suitable enzymes are described in other patents, for example WO2020/161480 and GB2012542.3.

The amino acid sequence of the spotted gar ( Lepisosteusoculatus) is shown below

MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

The inventors have identified various regions in the amino acid sequence having improved properties. Certain regions improve the solubility and handling of the enzyme. Certain other regions improve the ability to incorporate nucleotides with modifications at the 3ʹ-position.

Modifications which improve the solubility include a modification within the amino acid region WLLNRLINRLQNQGILLYYDIV shown highlighted in the sequence below.

MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

Modifications which improve the incorporation of modified nucleotides can be at one or more of selected regions shown below. The second modification can be selected from one or more of the amino acid regions VAIF, EDN, MGA, ENHNQ, FMRA, HAl, TKA, FHS, QADNA, MQK, SAAVCK, EAQA, TVR, KEC, TPEMGK, DHFQ, LAAG, APPVDN, FARHERKMLLDNHA, and YIDP shown highlighted in the sequence below.

MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

Modified terminal deoxynucleotidyl transferase (TdT) enzymes comprising at least one amino acid modification when compared to a wild type sequence SEQ ID NO 1 or the homologous amino acid sequence of a terminal deoxynucleotidyl transferase (TdT) enzyme in other species may be preferable for use in the methods described herein, wherein the modification is selected from one or more of the amino acid regions WLLNRLINRLQNQGILLYYDI, VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions.

Preferable sequences can contain both modifications, namely
a. a first modification is within the amino acid region WLLNRLINRLQNQGILLYYDI of the sequence of SEQ ID NO 1 or the homologous region in other species; and
b. a second modification is selected from one or more of the amino acid regions VAIF, EDN, MGA, ENHNQ, FMRA, HAl, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

As a comparison with other species, the sequence of Bostaurus (bovine) TdT is shown below:

MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

Modifications which improve the solubility include a modification within the amino acid region QLLPKVINLWEKKGLLLYYDLV shown highlighted in the sequence below.

MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

Modifications which improve the incorporation of modified nucleotides can be at one or more of selected regions shown below. The second modification can be selected from one or more of the amino acid regions LVLF, ENN, MGA, NNYNH, FMRA, FTI, VKC, FRS, MSDKT, MQK, EAEA, AVW, KKI, SPGSAE, DHFQ, MCPYEN, YATHERKMMLDNHA, and YIEP shown highlighted in the sequence below.

MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHEGKTWKAIRVDLVMCPYENRAFALLGWTGSRFERDIRRYATHERKMLDNHALYDKTKRVFLKAESEEEIFAHLGLDYIEPWERNA

As a comparison with other species, the sequence of Musmusculus (mouse) TdT is shown below:

MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

Modifications which improve the solubility include a modification within the amino acid region QLLHKVTDFWKQQGLLLYCDIL shown highlighted in the sequence below:

MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

Modifications which improve the incorporation of modified nucleotides can be at one or more of selected regions shown below. The second modification can be selected from one or more of the amino acid regions LVLF, ENN, MGA, NNYNQ, FMRA, FPI, VKS, FRT, QSDKS, MQK, VSCVNR, EAEA, AVV, KMT, SPEATE, DHFQ, MCPYDR, YATHERKMMLDNHA, and YIEP shown highlighted in the sequence below.

MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

Thus by a process of aligning sequences, it is immediately apparent which regions in the sequences of terminal transferases from other species correspond to the sequences described herein with respect to the spotted gar sequence shown in SEQ ID NO 1.

Modified terminal deoxynucleotidyl transferase (TdT) enzymes that may be used in the methods of the invention may comprise at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid regions WLLNRLINRLQNQGILLYYDI, VAIF, EDN, MGA, ENHNQ, FMRA, HAl, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

Furthermore, TdT enzymes that may be used in the methods of the invention include a modified TdT enzyme comprising at least two amino acid modifications when compared to a wild type sequence SEQ ID NO 1 or the homologous amino acid sequence of a terminal deoxynucleotidyl transferase (TdT) enzyme in other species, wherein;
a. a first modification is within the amino acid region WLLNRLINRLQNQGILLYYDIV of the sequence of SEQ ID NO 1 or the homologous region in other species; and
b. a second modification is selected from one or more of the amino acid regions VAIF, EDN, MGA, ENHNQ, FMRA, HAl, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species.

When compared to the sequence of Bostaurus (bovine) TdT; SEQ ID NO 2,
a. a first modification is within the amino acid region QLLPKVINLWEKKGLLLYYDLV of the sequence of SEQ ID NO 2 or the homologous region in other species; and
b. a second modification is selected from one or more of the amino acid regions LVLF, ENN, MGA, NNYNH, FMRA, FTI, VKC, FRS, MSDKT, MQK, EAEA, AVW, KKI, SPGSAE, MCP, YATHERKMMLDNHA, and YIEP of the sequence of SEQ ID NO 2 or the homologous regions in other species.

When compared to the sequence of Musmusculus (mouse) TdT; SEQ ID NO 3,
a. a first modification is within the amino acid region QLLHKVTDFWKQQGLLLYCDIL of the sequence of SEQ ID NO 3 or the homologous region in other species; and
b. a second modification is selected from one or more of the amino acid regions LVLF, ENN, MGA, NNYNQ, FMRA, FPI, VKS, FRT, QSDKS, MQK, VSCVNR, EAEA, AVV, KMT, SPEATE, DHFQ, MCPYDR, YATHERKMMLDNHA, and YIEP of the sequence of SEQ ID NO 3 or the homologous regions in other species.

The modifications can be chosen from any amino acid that differs from the wild type sequence. The amino acid can be a naturally occurring amino acid. The modified amino acid can be selected from ala, arg, asn, asp, cys, gln, glu, gly, his, ile, leu, lys, met, phe, pro, ser, thr, trp, val, and sec.

For the purposes of brevity, the modifications are further described in relation to SEQ ID NO 1, but the modifications are applicable to the sequences from other species, for example those sequences listed above having sequences in the NCBI database.

The sequences can be modified at positions in addition to those regions described. Embodiments of the invention may include for example methods that make use of sequences having modifications to amino acids outside the defined positions, providing those sequences retain terminal transferase activity. Embodiments of the invention may include for example methods that make use of sequences having truncations of amino acids outside the defined positions, providing those sequences retain terminal transferase activity. For example the sequences may be BRCT truncated as described in application WO2018215803 where amino acids are removed from the N-terminus whilst retaining or improving activity. Alterations, additions, insertions or deletions or truncations to amino acid positions are therefore within the scope of the methods of the invention.

The modification within the region WLLNRLINRLQNQGILLYYDIV or the corresponding region from other species help improve the solubility of the enzyme. The modification within the amino acid region WLLNRLINRLQNQGILLYYDIV can be at one or more of the underlined amino acids.

Particular changes can be selected from W-Q, N-P, R-K, L-V, R-L, L-W, Q-E, N-K, Q-K or I-L. The sequence WLLNRLINRLQNQGILLYYDIV can be altered to QLLPKVINLWEKKGLLLYYDLV.

The second modification improves incorporation of nucleotides having a modification at the 3’ position in comparison to the wild type sequence. The second modification can be selected from one or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species. The second modification can be selected from two or more of the amino acid regions VAIF, EDN, MGA, ENHNQ, FMRA, HAI, TKA, FHS, QADNA, MQK, SAAVCK, EAQA, TVR, KEC, TPEMGK, DHFQ, LAAG, APPVDN, FARHERKMLLDNHA, and YIDP of the sequence of SEQ ID NO 1 or the homologous regions in other species shown highlighted in the sequence below.

MLHIPIFPPIKKRQKLPESRNSCKYEVKFSEVAIFLLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

The identified positions commence at positions V32, E74, M108, F182, T212, D271, M279, E298, A421, L456, Y486. Modifications disclosed herein contain at least one modification at the defined positions.

The modified amino acid can be in the region FMRA. The modified amino acid can be in the region QADNA. The modified amino acid can be in the region EAQA. The modified amino acid can be in the region APP. The modified amino acid can be in the region LDNHA. The modified amino acid can be in the region YIDP. The region FARHERKMLLDNHA is advantageous for removing substrate biases in modifications. The FARHERKMLLDNHA region appears highly conserved across species.

The modification selected from one or more of the amino acid regions FMRA, QADNA, EAQA, APP, FARHERKMLLDNHA, and YIDP can be at the underlined amino acid(s).

The positions for modification can include A53, V68, V71, D75, E97, 1101, G109, Q115, V116, S125, T137, Q143, N154, H155, Q157, I158, 1165, G177, L180, A181, M183, A195, K200, T212, K213, A214, E217, T239, F262, S264, Q269, N272, A273, K281, S291, K296, Q300, T309, R311, E330, T341, E343, G345, N352, N360, Q361, 1363, Y367, H389, L403, G406, D411, A421, P422, V424, N426, R438, F447, R452, L455, and/or D488.

Amino acid changes include any one of A53G, V68I, V71I, D75N, D75Q, E97A, I101V, G109E, G109R, Q115E, V116I, V116S, S125R, T137A, Q143P, N154H, H155C, Q157K, Q157R, I158M, I165V, G177D, L180V, A181E, M183R, A195P, K200R, T212S, K213S, A214R, E217Q, T239S, F262L, S264T, Q269K, N272K, A273S, A273T, K281R, S291N, K296R, Q300D, T309A, R311W, E330N, T341S, E343Q, G345R, N352Q, N360K, Q361K, I363L, Y367C, H389A, L403R, G406R, D411N, A421L, A421M, A421V, P422A, P422C, V424Y, N426R, R438K, F447W, R452K, L455I, and/or D488P.

Amino acid changes include any two or more of A53G, V68I, V71I, D75N, D75Q, E97A, I101V, G109E, G109R, Q115E, V116I, V116S, S125R, T137A, Q143P, N154H, H155C, Q157K, Q157R, I158M, I165V, G177D, L180V, A181E, M183R, A195P, K200R, T212S, K213S, A214R, E217Q, T239S, F262L, S264T, Q269K, N272K, A273S, A273T, K281R, S291N, K296R, Q300D, T309A, R311W, E330N, T341S, E343Q, G345R, N352Q, N360K, Q361K, I363L, Y367C, H389A, L403R, G406R, D411N, A421L, A421M, A421V, P422A, P422C, V424Y, N426R, R438K, F447W, R452K, L455Iand/or D488P.

The modification of QADNA to KADKA, QADKA, KADNA, QADNS, KADNT, or QADNT is advantageous for the incorporation of 3ʹ-O-modified nucleoside triphosphates to the 3ʹ-end of nucleic acids and removing substrate biases during the incorporation of modified nucleoside triphosphates. The modification of APPVDN to MCPVDN, MPPVDN, ACPVDR, VPPVDN, LPPVDR, ACPYDN, LCPVDN, or MAPVDN is advantageous for the incorporation of 3ʹ-O-modified nucleoside triphosphates to the 3ʹ-end of nucleic acids and removing substrate biases during the incorporation of modified nucleoside triphosphates. The modification of FARHERKMLLDRHA to WARHERKMILDNHA, FARHERKMILDNHA, WARHERKMLLDNHA, FARHERKMLLDRHA, or FARHEKKMLLDNHA is also advantageous for the incorporation of 3ʹ-O-modified nucleoside triphosphates to the 3ʹ-end of nucleic acids and removing substrate biases during the incorporation of modified nucleoside triphosphates.

The modification can be selected from one or more of the following sequences FRRA, QADKA, EADA, MPP, FARHERKMLLDRHA, and YIPP. Included is a modified terminal deoxynucleotidyl transferase (TdT) enzyme wherein the second modification is selected from two or more of the following sequences FRRA, QADKA, EADA, MPP, FARHERKMLLDRHA, and YIPP. Included is a modified terminal deoxynucleotidyl transferase (TdT) enzyme wherein the second modification contains each of the following sequences FRRA, QADKA, EADA, MPP, FARHERKMLLDRHA, and YIPP.

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial nucleosides.

Therefore, references herein to ‘3ʹ-blocked nucleoside triphosphates’ refer to nucleoside triphosphates (e.g., dATP, dGTP, dCTP or dTTP) which have an additional group on the 3ʹ end which prevents further addition of nucleotides, i.e., by replacing the 3ʹ-OH group with a protecting group.

It will be understood that references herein to ‘3ʹ-block’, ‘3ʹ-blocking group’ or ‘3ʹ-protecting group’ refer to the group attached to the 3ʹ end of the nucleoside triphosphate which prevents further nucleotide addition. The present method uses reversible 3ʹ-blocking groups which can be removed by cleavage to allow the addition of further nucleotides. By contrast, irreversible 3ʹ-blocking groups refer to dNTPs where the 3ʹ-OH group can neither be exposed nor uncovered by cleavage.

References herein to ‘cleaving agent’ refer to a substance which is able to cleave the 3ʹ- blocking group from the 3ʹ-blocked nucleoside triphosphate. In one embodiment, the cleaving agent is a chemical cleaving agent. In an alternative embodiment, the cleaving agent is an enzymatic cleaving agent.

References herein to an ‘initiator oligonucleotide’ refer to a short oligonucleotide with a free 3ʹ-end which the 3ʹ-blocked nucleoside triphosphate can be attached to. In one embodiment, the initiator sequence is a DNA initiator sequence. In an alternative embodiment, the initiator sequence is an RNA initiator sequence.

References herein to a ‘DNA initiator sequence’ refer to a short DNA oligonuleotide with a free 3ʹ-end which the 3ʹ-blocked nucleoside triphosphate can be attached to, i.e., DNA will be synthesised from the end of the DNA initiator sequence.

In one embodiment, the initiator oligonucleotide is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long.

In one embodiment, the initiator oligonucleotide is single-stranded. In an alternative embodiment, the initiator oligonucleotide is double-stranded. It will be understood by persons skilled in the art that a 3ʹ-overhang (i.e., a free 3ʹ-end) allows for efficient addition.

The initiator oligonucleotide is immobilised on a solid support. This allows TdT and the cleaving agent to be removed between cycles of sequence extension without washing away the synthesised nucleic acid. The methods described herein may be carried out under aqueous conditions so that the methods can be easily performed via a flow setup.

In one embodiment, the terminal deoxynucleotidyl transferase (TdT) is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc. all with appropriate counterions, such as Cl) and inorganic pyrophosphatase (e.g., the Saccharomycescerevisiae or Escherichiacoli homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, step (c) is performed at a temperature less than 99° C., such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, after steps (b) and (c) a wash solution is applied. In one embodiment, the wash solution comprises the same buffers and salts as used in the extension solution described herein. This has the advantage of allowing the wash solution to be collected and recycled as extension solution in step (b) when the method steps are repeated.

In another embodiment, the wash solution contains agents to abolish secondary structure or protein-nucleic acid interactions. Suitable agents are known in the art to include monovalent salts, divalent salts, chaotropic agents such as guanidinium chloride, proteinase K, detergents, and surfactants.

Also disclosed is a kit suitable for carrying out the methods of the invention, wherein the kit comprises:
(i) a solid support, wherein the solid support is coated with a co-polymer to which an initiator oligonucleotide is attached, wherein the co-polymer is a co-polymer of a first co-monomer selected from acrylamide, methacrylamide, N-methylacrylamide, N,N′-dimethylacrylamide, hydroxyethyl methacrylate and N-vinyl pyrrolidinone and a second co-monomer which attaches the initiator oligonucleotide;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

Also disclosed is a kit comprising:
(i) a solid support, wherein the solid support comprises a co-polymer to which an initiator oligonucleotide is attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches the initiator;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

The method can be performed on a microfluidic device such as a digital microfluidic device.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWOD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

The oil in the device can be any water immiscible or hydrophobic liquid. The oil can be mineral oil, silicone oil, an alkyl-based solvent such as decane or dodecane, or a fluorinated oil. The air in the device can be any humidified gas.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever

Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

Magnetic particles of the invention can be used on a EWoD device and be retained in place on the device using magnets. Thus for example the beads can be retained on the surface of the support whilst the surrounding liquid is exchanged. Thus the immobilised solid support can be exposed to cycles of different reagents.

### Example 1

Comparison of different PAC-BRAC polymer coating strategies and their influence on enzymatic DNA synthesis efficiency

### Overview

Silica magbeads were subjected to a series of coating treatments with the same PAC-BRAC polymer formulation to determine whether the coating time, or the period of pre-polymerisation prior to coating, affected the following set of factors:

1. The efficiency of oligo immobilization to the coated magbeads.

2. The efficiency of N+1 enzymatic addition to oligos immobilized to the coated magbeads.

3. The efficiency of N+12 enzymatic addition to oligos immobilized to the coated magbeads.

A graphical overview of the experimental layout is shown in Scheme 1.

Scheme 1. Experimental layout

[image]

### Method

Magnetic particles: Silica-coated 1 µm magnetic particles (Alpha Nanotech, 50 mg/mL suspension) were used for all of the conditions described.

Particle washing: 7 mg (140 µL) of silica-coated 1 µm magnetic particles (Si-beads) were washed prior to PAC-BRAC polymer coating by sequentially washing in Decon90, milliQ water, NaOH, HCI, and milliQ water.

PAC-BRAC polymerisation: The PAC-BRAC polymer solution used to coat the washed Si-beads was prepared as follows. 10 mL of 0.8% acrylamide solution (Sigma PN: A4058-100ML) was degassed before addition of 165 µL 10% BRAC (N-(5-bromoacetamidylpentyl)acrylamide) in DMF. To this solution was added 11.5 µL TEMED (Sigma PN: T9281-25ML). Polymerisation was initiated by addition of 100 µL 5% potassium persulfate solution (Sigma PN: 216224). Solution was left to stand at RT until required for coating.

Si-bead coating with PAC-BRAC polymer solution: The MilliQ supernatant was removed and discarded from Si-beads. Si-beads were coated with PAC-BRAC polymer solution as outlined in Table 1. 167 µL aliquots of PAC-BRAC solution were used to resuspend each of the pellets, either immediately after PAC-BRAC polymerisation initiation (t0) or after a delay following initiation as shown in Table 1.

TABLE 1

Si-bead PAC-Brac coating conditions (PCM = Polymer Coated Magbead)

| Aliquot | Coating start (mins) | Coating incubation (mins) |
|---|---|---|
| PCM01 | t0 | 30 |
| PCM02 | t0 | 90 |
| PCM03 | t0 | 240 |
| PCM04 | t0 | 330 |
| PCM05 | t0+30 | 90 |
| PCM06 | t0+90 | 90 |
| PCM07 | t0+240 | 90 |

Washing of PAC-BRAC coated Si-beads: Each aliquot of coated PAC-BRAC-Si-beads (PMC01-07) was washed with phosphate buffer pH 7.0.

Oligo immobilization to PAC-BRAC-Si-beads: PAC-BRAC-Si-bead aliquots were resuspended in a 10 µM solution of oligo D197 in 10 mM KPi pH 7.0. The resulting bead suspension was incubated at 52° C. and 1000 rpm for 60 minutes.

Oligo D197 sequence
5ʹ T*T*T*TTTTTTTTTTTTTTTTTTTTTTTTTTTTTTTTTTTTUTTTT/icy3/TTTTT 3ʹ
* = internal phosphorothioate bond
U = UDG-cleavable 2dU base

Washing of oligo-PAC-BRAC-Si-beads: Each aliquot of oligo-PAC-BRAC-Si-beads was washed with a high salt buffer. Washed pellets were resuspended in 50% formamide/milliQ water and the suspension incubated at 37° C. for 30 minutes. After formamide treatment, oligo-immobilized bead aliquots were washed with a high salt buffer and finally resuspended in HEPES-KOH pH 7.2 ready for use in enzymatic synthesis reactions.

N+1 enzymatic synthesis reactions: Enzymatic 3ʹ-oxyamine N+1 nucleotide addition reactions were conducted on each of the oligo-PAC-BRAC-Si-bead aliquots; reactions were performed in duplicate for each bead type. 150 µg of each oligo-PAC-BRAC-Si-magbead type (PCM01-07) was pipetted into wells of a 96 well plate. Beads were pelleted and resuspended in nucleotide addition mix (NAM) before incubation at 37 for 5 minutes. Each well was washed twice with a high salt buffer to stop the synthesis reaction and remove reaction components. Oligonucleotide was removed from the bead with uracil DNA glycosylase (UDG). The oligonucleotide was analysed by PAGE and visualised on a Typhoon scanner.

N+12 enzymatic synthesis reactions: Enzymatic 3ʹ-oxyamine N+12 (ATCGATCGATCG) nucleotide addition reactions were conducted on each of the oligo-PAC-BRAC-Si-bead aliquots; reactions were conducted in duplicate for each bead-type. 150 µg of each oligo-PAC-BRAC-Si-magbead type (PCM01-07) was pipetted into wells of a 96 well plate. Beads were pelleted and resuspended in nucleotide addition mix (NAM) before incubation at 37 for 5 minutes. Each well was washed twice with a high salt buffer to stop the synthesis reaction and remove reaction components. Beads were pelleted and resuspended in nitrite deprotection solution (NDS) before incubation at room temperature for 5 minutes. Each well waas washed twice with a low salt buffer to stop the deprotection reaction and remove reaction components. The NAM, washing, NDS, washing process was repeated 12 times. Oligonucleotide was then recovered from the beads with uracil DNA glycosylase (UDG). The oligonucleotide was analysed by PAGE and visualised on a Typhoon scanner.

### Results

N+1 enzymatic synthesis reactions: FIG. 2 shows the PAGE image for N+1 addition reactions performed on different PAC-BRAC polymer coated bead types (PCM01-07). The intensity of bands clearly shows that certain bead treatments are beneficial. Plotting the N-band intensity of each bead type (FIG. 3) aids interpretation of the gel image.

### Example 2: Varying the Identity of the First Co-Monomer

Comparison of polymers formed from varying the first co-monomer while maintaining the identity of the second co-monomer.

### OVERVIEW

Silica paramagnetic particles were subjected to a series of coating treatments with different xPAC-BRAC polymer formulations, where xPAC indicates that different acrylamide monomers were used in the polymer preparation. The different coatings were performed to determine:

1. The ability to form polymer coats on solid supports with a range of monomer identities and combinations.

2. The performance of the various polymer coats in a method of enzymatic DNA synthesis.

### Method

Solid support preparation: The polymerisation and coating of solid support was performed as described in other examples, with the exception that some or all (0-100%) of the acrylamide co-monomer was replaced with either N-(hydroxylmethyl)acrylamide, N-(hydroxyethyl)acrylamide, N,N′-dimethyl acrylamide, or N-[tris(hydroxymethyl)methyl]acrylamide. Oligonucleotide initiator grafting was performed as described in a previous example.

N+17 enzymatic synthesis reactions: Enzymatic N+17 (CTTCATGACGTAAGGCC) nucleotide addition reactions were conducted on each of the oligo-xPAC-BRAC-Si-bead aliquots; reactions were conducted in duplicate for each bead-type. 150 µg of each oligo-PAC-BRAC-Si-magbead type was pipetted into wells of a 96 well plate. Beads were pelleted and resuspended in nucleotide addition mix (NAM; containing engineered TdT, 3ʹ-aminooxy nucleotide, divalent metal ion, inorganic pyrophosphatase, and buffered to pH 7.2) before incubation at 37° C. for 5 minutes. Each well was washed twice with a high salt buffer to stop the synthesis reaction and remove reaction components. Beads were pelleted and resuspended in nitrite deprotection solution (NDS) before incubation at room temperature for 5 minutes. Each well was washed twice with a low salt buffer to stop the deprotection reaction and remove reaction components. The NAM, washing, NDS, washing process was repeated 17 times. Oligonucleotide was then recovered from the beads with uracil DNA glycosylase (UDG). The samples were prepared into indexed libraries and sequenced on an Illumina iSeq. For each sample, the percentage of reads that showed synthesis of the correct length and sequence identity was calculated.

### Results

FIG. 7 shows the effect of altering the first co-monomer identity on polymer performance in a method of multicycling enzymatic synthesis. Complete replacement of acrylamide as the first co-monomer with N-[tris(hydroxymethyl)methyl]acrylamide or N,N′-dimethyl acrylamide reduces the synthesis performance obtained on the surface; meanwhile complete replacement with N-(hydroxylmethyl)acrylamide or N-(hydroxyethyl)acrylamide yielded a result comparable to the parent polymer (100% acrylamide as the first co-monomer). Surprisingly, using a mixed species first co-polymer (e.g. 50% acrylamide and 50% N-(hydroxymethyl)acrylamide) generates a polymer coating that exceeds the performance of the parent polymer. This situation can occur with even a small percentage addition of an alternative first co-monomer. Overall, this work demonstrates that the properties of the polymer coating can be finely tuned by either altering the identity of the first co-monomer or using a mixture of first co-monomers (i.e. a mixture of compounds that form the polymer but do not serve as grafting points for an initiator oligonucleotide).

### Example 3. Preparation of Oligonucleotides Using the Methods of the Invention in a Frit-Retained Beads Format

The oligo-PAC-BRAC-Si-beads can be used in a method of enzymatic synthesis whereby the solutions are exchanged by means of pelleting the beads using a magnet. However, there are other ways of exchanging the solutions. In this example, the oligo-PAC-BRAC-Si-beads are used in a method of enzymatic synthesis whereby the solutions are exchanged by means of retaining the polymer coated beads with a frit in a well and applying a vacuum to remove the solutions through the frit.

### Method

PAC-BRAC-Si-beads (100% acrylamide as first co-monomer) were prepared and had oligonucleotide initiators grafted to them as described in Example 1. Enzymatic DNA synthesis was performed on a Tecan Freedom Evo 200 liquid handling system using the following automated workflow (T-NAM = nucleotide addition mix [containing an engineered terminal deoxynucleotidyl transferase, divalent metal ion, inorganic pyrophosphatase, reversibly terminated dTTP nucleotide, and buffered to pH 7.2], wash 1 = high salt wash buffer, NDS = nitrite deprotection solution, wash 2 = low salt buffer):

| Step | Description | Stage |
|---|---|---|
| 1 | aliquot 100 µL bead solution (1.5 ug/ml) per well | Preparation |
| 2 | Apply 350 mBar pressure for 10 sec | |
| 3 | Apply 60 µL T-NAM per well onto filter plate and mix 5 times | Addition |
| 4 | Incubate at 37° C. for 15 minutes on shaker | |
| 5 | Pipette mix 5 times | |
| 6 | Apply 350 mBar pressure for 10 sec | |
| 7 | Apply 180 µL wash 1 per well onto filter plate and mix 5 times | |
| 8 | Apply 350 mBar pressure for 10 sec | |
| 9 | Repeat steps 7-8 twice | |
| 10 | Apply 180 µL wash 2 per well onto filter plate and mix 5 times | |
| 11 | Apply 350 mBar pressure for 10 sec | |
| 12 | Apply 60 µL NDS per well onto filter plate and mix 5 times | Deblock |
| 13 | Incubate at 25° C. for 5 min on shaker | |
| 14 | Apply 350 mBar vacuum pressure for 10 sec | |
| 15 | Pipette mix 5 times | |
| 16 | Apply 180 µL wash 2 per well onto filter plate and mix 5 times | |
| 17 | Apply 350 mBar pressure for 10 sec | |
| 18 | Repeat steps 19-20 twice | |

Repeat addition and deblock stages (i.e. steps 3-18) as required.

The synthesised oligonucleotides were then cleaved from the solid support using uracil DNA glycosylase (UDG) and alkaline cleavage of the resulting abasic site. The cleaved oligonucleotides were analysed by polyacrylamide gel electrophoresis (PAGE) and visualized by virtue of an internal TAMRA dye on a Typhoon Biomolecular Imager.

### Results

FIG. 8 shows the resulting gel image from representative wells from the 96-well and 384-well fritted plates. An initiator control was run in lane 1 as an N marker. The increase in molecular weight corresponding to the TdT-mediated addition of 8 nucleotides causes the product to migrate slower down the gel giving the major N+8 band (initiator + TTTTTTTT). These results clearly show that polymer coated beads support enzymatic synthesis in a fritted column format. Each well of the multiwell plate has a frit as the bottom surface that retains the polymer coated beads while solutions are dispensed into the wells and then removed by application of a vacuum. This set up is analogous to packed bed reactors commonly used in the chemical and biochemical industries.

### Example 4. Preparation of Oligonucleotides Using the Methods of the Invention in a Polymer Coated Glass Fibre Frit Format

The oligo-PAC-BRAC-Si-beads can be used in a method of enzymatic synthesis whereby the solutions are exchanged by means of pelleting the beads using a magnet. However, there are other ways of exchanging the solutions. In this example, the glass fibre frit of a multiwell plate is directly coated with pre-polymerised PAC-BRAC and subsequently oligonucleotide initiators are attached. Addition, wash, and deblock solutions are then conveniently exchanged by means of applying a vacuum.

### Method

Pre-polymerisation: The PAC-BRAC polymer solution was prepared as follows. First solution A was formed by combining 125 µL 40% w/v acrylamide solution with 10 mL of ultrapure water. Solution A was then degassed with nitrogen for 20 minutes. Solution B was prepared by dissolving 108.8 mg BRAC in 1080 µL dimethylformamide. Solution C was prepared by dissolving 177 mg potassium persulfate in 3527 µL ultrapure water. Solution B (800 µL) was added to solution A and vortexed to mix. To the combined solution A+B was added 11.5 µL tetramethylethylenediamine following by mixing. Polymerisation was initiated by addition of 100 µL of solution C and brief mixing. The polymerising solution was sealed and kept in the dark for 18 hours. Centrifugation was performed prior to decanting the polymer into a fresh tube.

### Glass Fibre Frit Preparation, Polymer Coating, and Initiator Grafting

Wells of an AcroPrep 384-well Glass Fibre Filter Plate (5073W) were treated in turn with 80% Decon, 1 M NaOH, 0.1 M HCl, and ultrapure water to clean the glass fibre frits. The pre-polymerised PAC-BRAC was applied to the glass fibre frits in 80 µL portions and incubated for 60 seconds before application of vacuum to remove the solution. This coating step was repeated four times. The polymer coated glass fibre frit was then washed with 80 µL potassium phosphate (10 mM, pH 7) three times. Oligonucleotide initiator containing three internal phosphorothioate linkages was grafted to the polymer through 30 minutes of incubation at 37° C. Excess initiator was washed from the frit with 20 mM NaOH. Finally the plates were reconditioned with potassium phosphate buffer (10 mM, pH 7).

### Enzymatic DNA Synthesis

Enzymatic DNA synthesis was performed on a Tecan Freedom Evo 200 liquid handling system using the following automated workflow (T-NAM = nucleotide addition mix [containing an engineered terminal deoxynucleotidyl transferase, divalent metal ion, inorganic pyrophosphatase, reversibly terminated dTTP nucleotide, and buffered to pH 7.2], wash 1 = high salt wash buffer, NDS = nitrite deprotection solution, wash 2 = low salt buffer):

| Step | Description | Stage |
|---|---|---|
| 1 | Apply 30 µL T-NAM per well onto filter plate | Addition |
| 2 | Apply 690 mBar vacuum for 0.1 sec | |
| 3 | Incubate at 37° C. for 15 | |
| 4 | Apply 250 mBar pressure for 20 sec | |
| 5 | Apply 80 µL wash 1 per well onto filter plate | |
| 6 | Apply 250 mBar pressure for 20 sec | |
| 7 | Repeat steps 7-8 | |
| 8 | Apply 80 µL wash 2 per well onto filter plate | |
| 9 | Apply 250 mBar pressure for 60 sec | |
| 10 | Repeat steps 10-11 | |
| 12 | Apply 50 µL NDS per well onto filter plate | Deblock |
| 13 | Apply 690 mBar vaccum for 0.05 sec | |
| 14 | Incubate at room temperature for 3 minutes | |
| 15 | Apply to 250 mBar for 20 sec | |
| 16 | Repeat 12-15 twice | |
| 17 | Repeat steps 7-8 twice | |
| 18 | Repeat steps 8-9 | |

Repeat add t on and debloc< stages (i.e. steps 3-18) as required.

The synthesised oligonucleotides were then cleaved from the solid support using uracil DNA glycosylase (UDG) and N,N′-dimethylethylenediamine (DMED). The cleaved oligonucleotides were analysed by polyacrylamide gel electrophoresis (PAGE) and visualized by virtue of an internal TAMRA dye on a Typhoon Biomolecular Imager.

### Results

FIG. 9 shows the resulting gel image from representative wells from the 384-well glass fibre fritted plates. An initiator control was run in lane 1 as an N marker. The increase in molecular weight corresponding to the TdT-mediated addition of 8 nucleotides causes the product to migrate slower down the gel giving the major N+8 band (initiator + TTTTTTTT). These results clearly show that polymer coated beads support enzymatic synthesis in a fritted column format. Each well of the multicell plate has a frit as the bottom surface that maintains the polymer coated beads while solutions are dispensed into the wells and then removed by application of a vacuum. This set up is analogous to the packed bed reactors commonly used in the chemical and biochemical industries.

1. A method of nucleic acid synthesis, wherein the method comprises the steps of:
(a1) providing a solid support comprising particles coated with a pre-polymerised material, wherein the pre-polymerised material comprises a co-polymer to which an initiator oligonucleotide is to be attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches to the initiator;
(a2) coupling an initiator oligonucleotide to said co-polymer to form a solid-supported initiator oligonucleotide;
(b) adding a 3ʹ-blocked nucleoside triphosphate to said initiator oligonucleotide in the presence of a terminal deoxynucleotidyl transferase (TdT) enzyme or modified terminal deoxynucleotidyl transferase (TdT) enzyme;
(c) cleaving the blocking group from the 3ʹ-blocked nucleoside triphosphate in the presence of a cleaving agent; and
(d) repeating steps (b) and (c) to synthesise an extended nucleic acid.

2. The method according to claim 1, wherein the first co-monomer is acrylamide.

3. The method according to any one of claims 1 or 2, wherein the solid support is coated with a pre-polymerised material comprising a monomer selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer containing a reactive moiety selected from haloacetamide, carboxylic acid, alkyne, azide, amine or thiol.

4. The method according to claim 3, wherein the second co-monomer is a haloacetamide-containing monomer.

5. The method according to any one of claims 1 to 4, wherein the second co-monomer is a monomer of formula (3) or (3a): 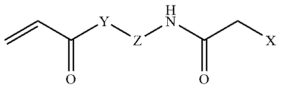 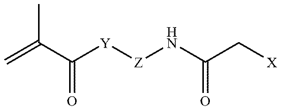
wherein X is Cl, Br or I;
Y is NR¹ or 0;
Z is an optionally substituted C₁₋₅₀ alkyl bridge;
and R¹ is H or an optionally substituted C₁₋₅ alkyl group.

6. The method according to claim 5, wherein X is Br.

7. The method according to claim 5 or 6, wherein Y is NH.

8. The method according to any one of claims 5 to 7, wherein Z is a C5 alkyl bridge.

9. The method according to any one of claims 1 to 8, wherein the initiator oligonucleotide is coupled via a phosphorothioate moiety.

10. The method according to any one of claims 1 to 8, wherein the initiator oligonucleotide is coupled via click chemistry between an azide and an alkyne.

11. The method according to any one of claims 1 to 10, wherein the extended nucleic acid is detached from the solid support.

12. The method according to any one of claims 1 to 11, wherein the initiator contains a uracil moiety and nucleic acid is detached by removing the uracil base and cleaving the abasic site.

13. The method according to any one of claims 1 to 12, wherein the particles are magnetic.

14. The method according to any one of claims 1 to 13, wherein the particles are silica coated.

15. The method as defined in any one of claims 1 to 14, wherein the 3ʹ-blocked nucleoside triphosphate is blocked a group selected from 3ʹ-O-azidomethyl, 3ʹ-aminooxy, 3ʹ-O-allyl, 3ʹ-O-cyanoethyl, 3ʹ-O-acetyl, 3ʹ-O-nitrate, 3ʹ-O-phosphate, 3ʹO-acetyl levulinic ester, 3ʹ-O-tert butyl dimethyl silane, 3ʹ-O-trimethyl(silyl)ethoxymethyl, 3ʹ-O-ortho-nitrobenzyl, or 3ʹ-O-para-nitrobenzyl.

16. The method according to any one of claims 1 to 15, wherein the cleaving agent is tris(2-carboxyethyl)phosphine (TCEP), a palladium complex, an organic or inorganic base, sodium nitrite or a photoactivated transition metal complex.

17. The method according to any one preceding claim, wherein the solid support contains a plurality of coagulated magnetic particles.

18. The method according to any one preceding claim, wherein the pre-polymerisation is carried out for at least 90 minutes prior to exposure to the surface being coated.

19. A kit comprising:
(i) a solid support comprising particles coated with a pre-polymerised material, wherein the pre-polymerised material comprises a co-polymer to which an initiator oligonucleotide is attached or is to be attached, wherein the co-polymer is a co-polymer of one or more first co-monomer(s) selected from acrylamide, methacrylamide, N-methylacrylamide, N,N′-dimethylacrylamide, N-(hydroxylmethyl)acrylamide, N-(hydroxyethyl)acrylamide, N-[tris(hydroxymethyl)methyl]acrylamide, hydroxyethyl methacrylate and N-vinyl pyrrolidinone and a second co-monomer which attaches to or is attached to the initiator;
(ii) a 3ʹ-blocked nucleoside triphosphate;
(iii) a terminal deoxynucleotidyl transferase (TdT) enzyme or modified terminal deoxynucleotidyl transferase (TdT) enzyme and optionally;
(iv) a cleaving agent.

20. The kit according to claim 19 further comprising an initiator oligonucleotide.

21. The kit according to claim 19, wherein the initiator oligonucleotide is attached to the coated support.
